(19) United States
(12) Patent Application Publication
(10) Pub. No.: US 2022/0322702 A1
(43) Pub. Date: Oct. 13, 2022

(54) SEAWEED BLEND FEED SUPPLEMENT

(71) Applicant: OCEAN HARVEST TECHNOLOGY (UK) LIMITED, Fareham (GB)

(72) Inventors: Graham Ellis, Fareham (GB); Jason Sands, Fareham (GB)

(21) Appl. No.: 17/622,406

(22) PCT Filed: Jun. 11, 2020
(86) PCT No.: PCT/GB2020/051405

§ 371 Date: Dec. 23, 2021

(30) Foreign Application Priority Data
Jul. 1, 2019 (GB) 1909443.2

(51) Int. Cl. A23K 10/30; A23K 50/20; A23K 50/10; A23K 50/70; A23K 50/30; A23K 50/40

(52) U.S. Cl. CPC A23K 10/30; A23K 50/20; A23K 50/10; A23K 50/70; A23K 50/30; A23K 50/40

(57) ABSTRACT

Use of a seaweed blend to modify gastrointestinal (GI) microbiota of an animal host. Modifying the GI microbiota may comprise modifying a community of bacteria, wherein the community of bacteria comprises Firmicutes and Bacteriodetes and the seaweed blend modifies the ratio of Firmicutes to Bacteriodetes, such as increasing the ratio of Firmicutes to Bacteriodetes. Further, the seaweed blend modifies specific families of commensal bacteria families including Ruminococceae and Lachnospiraceae within the Phyla Firmicutes. The seaweed blend may comprise (i) 65-80 wt % Ulva; (ii) 3-8 wt % Gracilaria; and (iii) 15-25 wt % Sargassum and/or Ascophyllum.

The present invention relates to the use of a seaweed blend as a feed supplement, particularly for domesticated animals.

### BACKGROUND TO THE INVENTION

An animal's gastrointestinal (GI) tract hosts a diversity of bacteria that form a stable community, with each bacterial species occupying its own ecological niche. The GI tract bacterial community varies depending on many factors including the animal species, the animal age, the location in the GI tract and dietary changes.

Probiotics and prebiotics have been proposed to alter GI microbiota. Probiotics are live microorganisms intended to provide health benefits when consumed, generally by improving or restoring the gut flora. Prebiotics are not live microorganisms, and instead induce the growth or activity of such commensal or “friendly” microorganisms.

Known prebiotics include fructans, which are commonly derived from chicory and Jerusalem artichoke. WO2015075440 proposes a prebiotic composition comprising a fructan extract derived from grasses.

### SUMMARY OF THE INVENTION

According to a first aspect of the invention there is provided the use of a seaweed blend to modify gastrointestinal (GI) microbiota, specifically butyrate producing bacterial species of an animal host.

In particular, the invention resides in the use of different blends of seaweeds in order to modify the GI microbiota of different animals. By adjusting the seaweed blend composition and/or the dose rate of the seaweed blend it is possible to target differing response levels within a specific animal host species, e.g. dialing up and down the levels of the polysaccharides.

The inventors have determined that the seaweed blend of the invention provides surprising benefits when employed as a feed supplement to modify GI microbiota of a host, in particular the GI microbiota of domesticated animals. GI microbiota, also known as gut microbiota or gut flora, is the complex community of microorganisms that live in the digestive tracts of animals, including humans.

Seaweed, or macroalgae, refers to macroscopic, multicellular, marine algae. The term includes Rhodophyta (red), Phaeophyta (brown) and Chlorophyta (green) macroalgae.

WO2015075440 recognises that there are drawbacks to the use of common sources of prebiotics, such as chicory and Jerusalem artichokes, and proposes the use of high-sugar grasses. The high sugar grasses are said to be a cheap and abundant source of prebiotic fructans which grow in a wider range of geographies than traditional sources of prebiotic fructans (chicory and Jerusalem artichokes). They are easier to harvest (not requiring the removal of the whole plant) and since they are not a food crop, they can be cheaper for manufacturers to purchase in bulk. While the grasses provide some benefits compared to common sources of prebiotics, they still suffer some disadvantages. In particular, the grasses must be juiced to provide a fructan extract having a useful amount of prebiotic.

The seaweed blend employed in the present invention has benefits relative to other sources of prebiotics. For example, seaweeds have high levels of fermentable prebiotic fibre, and have virtually no lignin, so deliver much lower levels of insoluble fibre—which has quite a different purpose in the gut.

According to a second aspect of the invention there is provided a method comprising providing a feed supplement comprising a seaweed blend to an animal host, the animal host having a gastrointestinal (GI) microbiota; and modifying the gastrointestinal (GI) microbiota of the animal.

The seaweed blend has a composition and is delivered to the animal host at a dosage (e.g. at a given rate such as daily at 0.5 wt % of total feed). The composition and the dosage of the feed supplement can be selected in order to target a specific desired response.

According to a third aspect of the invention there is provided a method to modify gastrointestinal (GI) microbiota of an animal host, the method comprising determining the relative abundance of at least one microorganism in a sample obtained from the host; and providing a feed supplement comprising a seaweed blend to the animal host, wherein the composition and/or dosage of the seaweed blend is selected based on the relative abundance of the at least one microorganism in the sample.

Suitable samples include a faecal sample or a caecal sample.

### DETAILED DESCRIPTION OF THE INVENTION

### Benefits

The invention resides in the use of a seaweed blend to modify the GI microbiota of an animal host. The GI microbiota is the complex community of microorganisms that live in the digestive tract, and includes bacteria, fungi, archaea, and viruses.

Modification of the GI microbiota includes increasing or decreasing the relative abundance of certain species of microorganisms, in particular species that are producers of butyrate and/or the total number of microorganisms. The relative abundance of a species relates to the proportion of that species in a sample, rather than the total population.

It will be understood that the total population and the composition of the population will differ based on factors including the host species, geography, season, diet, genetics etc. In any case, when the commensal or beneficial bacteria become more dominant, there is a prebiotic effect. While relative abundance is measured in the examples, the seaweed blend is also likely having an effect on the total number of each bacterium as well.

The present invention is primarily concerned with modification of GI bacteria. The examples measure all known microorganisms identified by 16S rRNA gene sequencing, in a sample.

Bacteria can be described with reference to their phylum, class, order, family, genus and species.

Dominant bacterial phyla in the GI tract include Firmicutes, Bacteroidetes, Actinobacteria, and Proteobacteria.

Firmicutes includes the genera Butyricicoccus, Clostridium, Lactobacillus, Faecalibacterium, Eubacterium, Ruminococcus, Coprococcus. Bacteroidetes includes the genera Bacteroides and Prevotella. Actinobacteria includes the genus Rubrobacter. Proteobacteria includes the genera Escherichia and Succinivibrio.

The seaweed blend may modify the ratio of Firmicutes:Bacteroidetes, such as increasing the ratio of Firmicutes:Bacteroidetes. Current perception is that microbiota establishment is regulated by the metabolic niche (mainly diet and antimicrobials), host genetic background, microbes-microbes interactions and host-microorganism interplay (Spor et al., 2011; Schloss et al., 2012; Bearson et al., 2013).

This may be beneficial since an increase in faecal Firmicutes has been reported to be associated with an increase in nutrient absorption, whereas an increase in faecal Bacteroidetes has been associated with a decrease in nutrient absorption (Jumpertz et al., 2011).

As shown in the examples, the use of the seaweed blend increased the ratio of Firmicutes to Bacteroidetes in the caeca of 6 week old broiler birds fed the seaweed blend, as compared to a control.

As shown in the examples, the use of the seaweed increased the ratio of Firmicutes to Bacteroidetes in the faeces of pigs fed the seaweed blend, as compared to a control.

Genera of bacteria include Bacteroides, Clostridium, Faecalibacterium, Eubacterium, Ruminococcus, Peptococcus, Peptostreptococcus, Bifidobacterium, Prevotella, Butyricicoccus, Coprococcus, Prevotella, Escherichia and Lactobacillus.

The seaweed blend may modify the relative abundance (e.g. increase the relative abundance) of one or more of Faecalibacterium, Bifidobacterium, Butyricicoccus, Coprococcus, and Lactobacillus.

The seaweed blend may modify the relative abundance of species of the genus Faecalibacterium. The seaweed blend may increase the relative abundance of Faecalibacterium. Faecalibacterium is a butyrate producing bacterial genus associated with carbohydrate metabolism. Butyrate has been shown to be anti-inflammatory (Van Immerseel et al., 2010; Ceiasco et al., 2014).

As shown in the examples, the relative abundance of the genus Faecalibacterium in the caeca of broiler birds fed diets containing the seaweed blend was higher than a control (no seaweed blend).

The seaweed blend may modify the relative abundance of species of the genus Bifidobacterium. The seaweed blend may increase the relative abundance of Bifidobacterium.

As shown in the examples, the relative abundance of Bifidobacterium species in the caeca of broiler birds fed diets containing the seaweed blend was higher than a control.

The seaweed blend may modify the relative abundance of species of the genus Lactobacillus. The seaweed blend may increase the relative abundance of Lactobacillus. Lactobacillus species such as L. fermentum have been used as a growth-promoting feed supplement preventing and treating diarrhoea of weaned piglets and maximising average daily gain, crude protein apparent digestibility and serum specific IgG level (Yu et al., 2008).

As shown in the examples, the relative abundance of Lactobacillus species in the faeces of pigs fed diets containing the seaweed blend was higher than a control.

In caeca of poultry lactobacillus is not a dominant genus and is not consistently increased the when birds are supplemented with seaweed blends. This is due to the anearobic environment of the caeca and the fact that Lactobacillus is not a strict anerobe. In swine, a sample of fresh faeces is typically used to sample the microbiota in the colon. An increase in lactobacillus is seen in the faeces of swine supplemented with seaweed prebiotics.

The seaweed blend may modify the relative abundance of species of the genus Butyricicoccus. The seaweed blend may increase the relative abundance of Butyricicoccus. Butyricicoccus and Blautia have been positively correlated with sIgA concentrations (Mach et al., 2015). These are butyrate producing species, which are known to be associated with reduction in enterocyte inflammation (Bedford and Gong, (2018).

As shown in the examples, the relative abundance of Butyricicoccus species in the faeces of pigs fed diets containing the seaweed blend was higher than a control.

The seaweed blend may modify the relative abundance of species of the genus Coprococcus. The seaweed blend may increase the relative abundance of Coprococcus. Coprococcus has been positively correlated with body weight post weaning (Mach et al., 2015).

As shown in the examples, the relative abundance of Coprococcus species in the faeces of pigs fed diets containing the seaweed blend was higher than a control.

The seaweed blend may modify the relative abundance of species of the genus Prevotella. The seaweed blend may reduce the relative abundance of Prevotella. Prevotella species are positively correlated with the weaning transition in young pigs. The increase in the relative abundance may be the result of diet-induced dysbiosis associated with the change to a grain-based diet and stress during the weaning transition (Gresse et al., Trends in Microbiology June 2017, http://dx.doi.org/10.1016/j.tim 0.2017.05.004).

The relative abundance of a particular species can be determined by analysing a sample from the GI tract such as a faecal sample or a caecal sample (from the caecum). The sample may be analysed by microbial profiling. For bacteria the sample may be analysed by 16S profiling, such as 16S RNA qPCR, cloning and sequencing (BaseClear NV). For fungi, the sample may be analysed by ITS (BaseClear NV). The microbial profiling may provide a table containing all taxonomies found and their counts.

Seaweed Blend

As noted above, seaweed, or macroalgae, refers to macroscopic, multicellular, marine algae. The term includes Rhodophyta (red), Phaeophyta (brown) and Chlorophyta (green) macroalgae.

The seaweed blend may comprise a blend of a green seaweed (e.g. Ulva), a brown seaweed (e.g. Sargassum or Ascophyllum) and a red seaweed (e.g. Gracilaria). The seaweed blend may comprise 30 to 95 wt % (e.g. 50 to 90 wt % or 60 to 75 wt %) green seaweed, 5 to 50 wt % (e.g. 10 to 30 wt % or 15 to 25 wt %) brown seaweed and 1 to 50 wt % (e.g. 2 to 20 wt % or 3 to l0 wt %) red seaweed. All values are given on a dry weight basis.

Seaweed can be described by reference to its genus. Seaweed genera include Ulva (green), Sargassum (brown), Fucus (brown), Gracilaria (red), Ascophyllum (brown), Laminaria (brown), Macrocystis (brown), Monostroma (green) and Porphyra (green).

The seaweed blend may comprise seaweed from at least two, at least three or at least four different genera.

The Ulva genus includes Ulva lactuca, known by the common name sea lettuce. The seaweed blend may comprise at least 10 wt %, at least 20 wt %, at least 30 wt %, at least 40 wt %, at least 50 wt %, at least 60 wt % or at least 70 wt % Ulva and/or the seaweed blend may comprise 85 wt % or less, 80 wt % or less, 75 wt % or less, 50 wt %, or 30 wt % or less Ulva.

The Sargassum genus includes Sargassum muticum (species), known by the common name Japanese wireweed. The seaweed blend may comprise at least 1 wt %, at least 3 wt %, at least 5 wt %, at least 10 wt % or at least 15 wt % Sargassum and/or the seaweed blend may comprise 50 wt % or less, 30 wt % or less, 20 wt % or less, 10 wt % or less or 5 wt % or less Sargassum. The seaweed blend may contain 0% sargassum.

The genus Ascophyllum includes Ascophyllum nodosum. The seaweed blend may comprise at least 3 wt %, at least 5 wt %, at least 8 wt %, at least 10 wt %, at least 15 wt %, at least 20 wt %, at least 30 wt %, at least 40 wt % or at least 50 wt % Ascophyllum and/or the seaweed blend may comprise 60 wt % or less, 50 wt % or less, 40 wt % or less, 30 wt % or less, 25 wt % or less, 20 wt % or less, 12 wt % or less or 8 wt % or less Ascophyllum.

The genus Gracilaria includes the species: Gracilaria bursa-pastoris, Gracilaria multipartite, Gracilaria gracilis, Gracilariopsis longissimi, Gracilaria verrucosa and Gracilaria confervoides. The seaweed blend may comprise at least 2 wt % at least 3 wt % or at least 5 wt % Gracilaria and/or the seaweed blend may comprise 20 wt % or less, 12 wt % or less or 8 wt % or less Gracilaria.

The seaweed blend may comprise Lithothamnion corallioides, Lithothamnion glaciale and/or Phymatolithon calcareum, commonly referred to as “Maerl”.

Specific seaweed blends are set out in the tables below.

| | Recipe A | Recipe B | Recipe C | Recipe D |
|---|---|---|---|---|
| | (wt %) | (wt %) | (wt %) | (wt %) |
| Ulva lactuca | 70-80 | 70-80 | 65-75 | 55-70 |
| Sargassum | 2-10 | 10-20 | 15-25 | 2-9 |
| Gracilaria | 3-8 | 3-8 | 2-15 | 3-10 |
| Ascophyllum | 10-20 | 3-8 | 2-15 | 15-25 |
| nodosum | | | | |
| Maerl | 0-1 | 0-3 | 0-7 | 0-7 |

| | Recipe A’ | Recipe B’ | Recipe C’ | Recipe D’ |
|---|---|---|---|---|
| | (wt %) | (wt %) | (wt %) | (wt %) |
| Ulva lactuca | 70-80 | 70-80 | 65-75 | 55-70 |
| Ascophyllum and | 2-20 | 3-20 | 2-25 | 2-25 |
| Sargassum | | | | |
| Gracilaria | 3-8 | 3-8 | 2-15 | 3-10 |
| Maerl | 0-1 | 0-3 | 0-7 | 0-7 |

The seaweed blend may comprise (i) Ulva, (ii) Gracilaria, (iii) Sargassum and/or Ascophyllum. For example, the seaweed blend may comprise (i) 65-80 wt % Ulva, (ii) 3-8 wt % Gracilaria, and (iii) 15-25 wt % Sargassum and/or Ascophyllum.

Selection of Composition and/or Dosage of the Seaweed Blend

The composition and/or dosage of the seaweed blend can be selected based on the desired modification of the microbiota. As such, the composition and/or dosage of the seaweed blend can be selected based on a number of factors including the host species, the host age, season, diet, genetics etc.

The examples demonstrate two different seaweed blends in two different animal species (broiler chicken and swine). Both seaweed blends provide an increase relative to a control of the ratio of Firmicutes to Bacteroides, which is considered beneficial in terms of nutrient absorption. The chicken trial shows an increase in relative abundance of Bifidobacterium and Faecalbacterium. The swine trial shows an increase in Lactobacillus, Butyricicoccus and Coprococcus.

Hence, a seaweed blend may be selected to increase relative abundance of Bifidobacterium and/or Faecalbacterium, for example in birds.

Hence a seaweed blend may be selected to increase relative abundance of Lactobacillus, Butyricicoccus and/or Coprococcus, for example in swine.

Feeding Regime

Where the seaweed blend feed supplement is for human consumption, it might be better described as a food supplement. The food supplement can be consumed on its own, as a tablet or capsule for example. Alternatively the food supplement may be combined with a foodstuff. The seaweed blend can be incorporated into a range of products including baked goods, dairy products, and confectionery. The seaweed blend serves as a prebiotic and can have benefits relative to probiotics, which tend to degrade when processed, for example at high temperature.

The seaweed blend may be provided to animals (e.g. domesticated animals) together with their regular feed (e.g. incorporated into a feed composition) or it may be provided separately from their regular feed. The seaweed blend may be incorporated into feed pellets. The feed pellets may be mixed into a diet on site (e.g. at a farm), or fed separately.

The amount used can be described relative to the amount of feed (food). The seaweed blend may be fed to an animal in an amount that is at least 0.2 wt %, at least 0.3 wt %, at least 0.5 wt %, at least 0.7 wt %, at least 1.0 wt %, at least 1.5 wt % or at least 2.0 wt % of the regular feed and/or the seaweed blend may be fed to the animal in an amount that is 5 wt % or less, 3 wt % or less, 2 wt % or less 1.5 wt % or less of the regular feed.

The seaweed blend may be provided ad libitum to allow the animal to consume the supplement as desired. Alternatively, the seaweed blend may be provided at regular intervals, such as once as day; every other day; or one a week.

The seaweed blend may be provided for at least 1 week, at least 4 weeks, at least 8 weeks, at least 12 weeks, at least 24 weeks, at least 36 weeks, at least 48 weeks or at least 60 weeks and/or the seaweed blend may be provided for 70 weeks or less, 50 weeks or less, 30 weeks or less, 20 weeks or less or 10 weeks or less.

The seaweed blend may be employed in the absence of antibiotics and/or coccidiostat. The seaweed blend may be incorporated into a feed composition.

Host

The host may be a non-human animal, especially domesticated animals including livestock and companion animals (pets). Alternatively, the host may be human.

The host may be selected from birds, swine (pigs), ruminants (including sheep and cattle), horses, and companion animals. Suitable birds include chickens, ducks, quail, goose, turkey, pheasant, guineafowl, and ostrich. Companion animals include dogs and cats.

The host may be a broiler bird, a bird bred primarily for meat production, such as Bresse, Cornish (a.k.a. Indian Game, including Cornish Cross), Ixworth and Jersey Giant.

The seaweed blend may be provided to the host from birth, from 4 weeks of age, or from 8 weeks of age, such as from 0 to 6 weeks of age or from 3 to 9 weeks of age.

Where the host is female (e.g. a sow), the seaweed blend may be provided to the host (e.g. swine) during gestation and/or lactation.

The invention will now be described, in a non-limiting fashion, with reference to the following figures:

FIGS. 1 to 3 show the relative abundance of various bacteria in the caeca of broiler chickens fed diets with and without a seaweed blend.

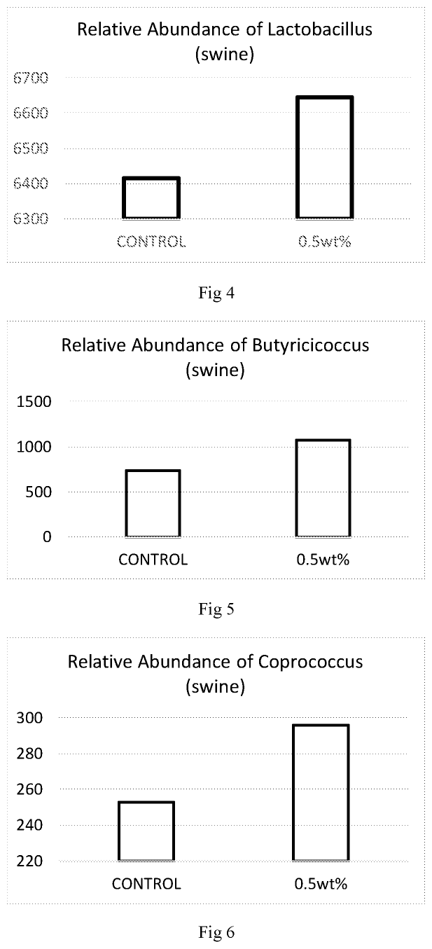

FIGS. 4 to 7 show the relative abundance of various bacteria in the faeces of pigs fed diets with and without a seaweed blend.

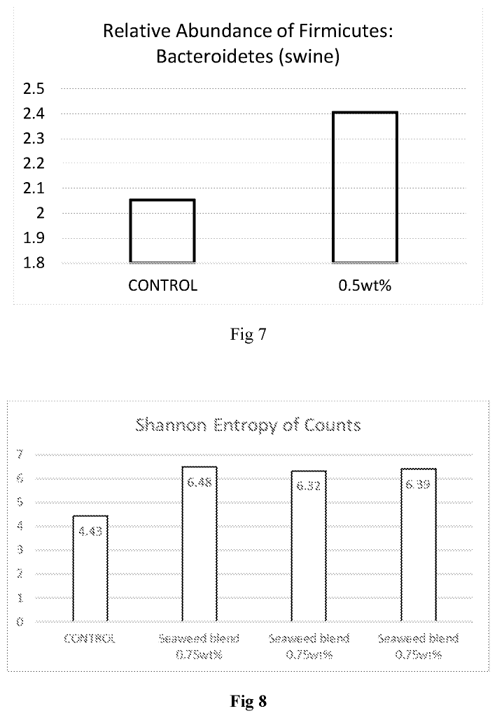

FIGS. 8 and 9 show Shannon Entropy of Counts (Alpha Diversity metric indicating higher diversity of bacteria) and the Firmicutes to Bacteroidetes ratio as identified by 16S RNA gene sequencing of faecal samples from pigs fed a Control diet or the Control with added seaweed blend.

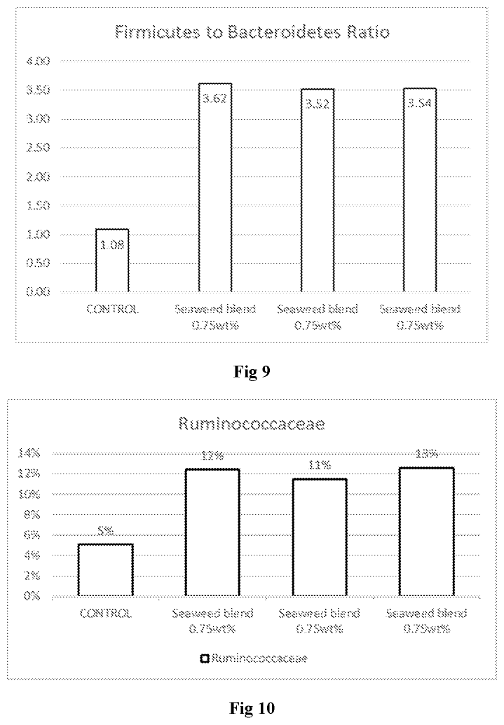

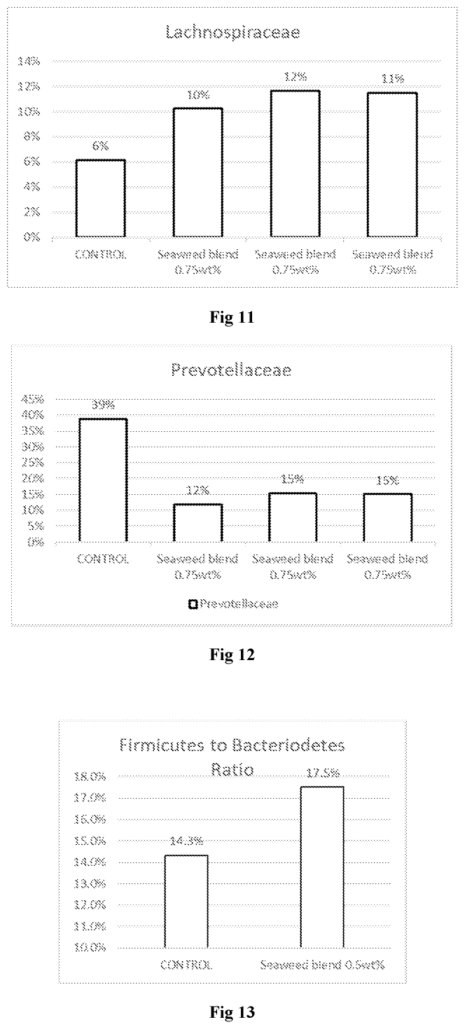

FIGS. 10, 11 and 12 show Relative Abundance of bacterial families Ruminococcaceae Prevotellaceae and Lachnospiraceae sampled at day 42 from the ceca of broiler chickens fed a control diet or the control diet supplemented with 0.5% Seaweed blend from 0-42 days of age. Significant increase in Ruminococcaceae (P=0.054).

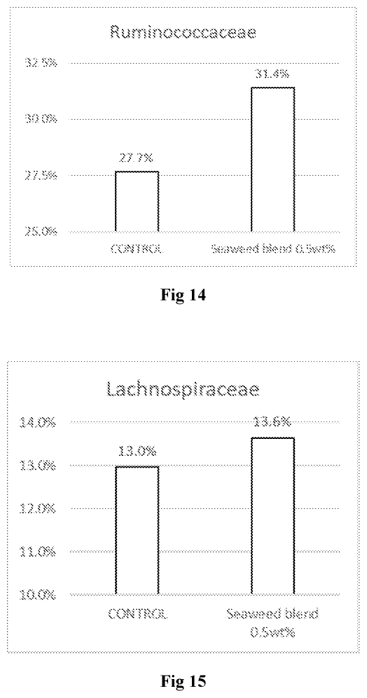

FIG. 13 shows Firmicutes to Bacteroidetes ratio as identified by 16S RNA gene sequencing of the ceca of broiler chickens fed a control diet or the control diet supplemented with 0.5% seaweed blend from 0-42 days of age.

FIGS. 14 and 15 show Relative Abundance of bacterial families Ruminococcaceae and Lachnospiraceae sampled at day 42 from the ceca of broiler chickens fed a control diet or the control diet supplemented with 0.5% from 0-42 days of age. Significant increase in Ruminococcaceae (P=0.054).

### BROILER CHICKEN TRIAL 1

Several studies have shown that growth performance, feed efficiency, and gut health in broiler chickens can be improved by dietary prebiotics, non-digestible carbohydrates selectively stimulating the growth of beneficial bacteria (Xu et al., 2003; Yusrizal & Chen, 2003; Yang et al., 2008). For example, feed supplementation with 0.4% fructo-oligosaccharides (FOS) in broiler chickens significantly increased body weight gain, feed efficiency, the activities of protease and amylase, ileal villus height, and the growth of Bifidobacterium and Lactobacillus (Xu et al., 2003).

| Recipe B | Wt % |
|---|---|
| Ulva lactuca | 70-80 |
| Sargassum | 10-20 |
| Gracilaria | 3-8 |
| Ascophyllum nodosum | 3-8 |
| Maerl | 0-3 |

A seaweed blend (above) was added to the feed (0.5 wt % of the feed) of commercial broilers from day 0 to 6 weeks of age, at which time caecal samples were collected to assess changes in the bacterial community. The cecum is an intraperitoneal pouch that is considered to be the beginning of the large intestine. Caecal samples were analysed by 16S RNA qPCR, cloning and sequencing (BaseClear NV).

Referring to FIG. 1, it can be seen that the relative abundance of bifidobacteria from the caeca of broiler chickens fed diets containing the seaweed blend was higher (27% compared to 25%) compared to control birds fed the same diet without the addition of the seaweed blend.

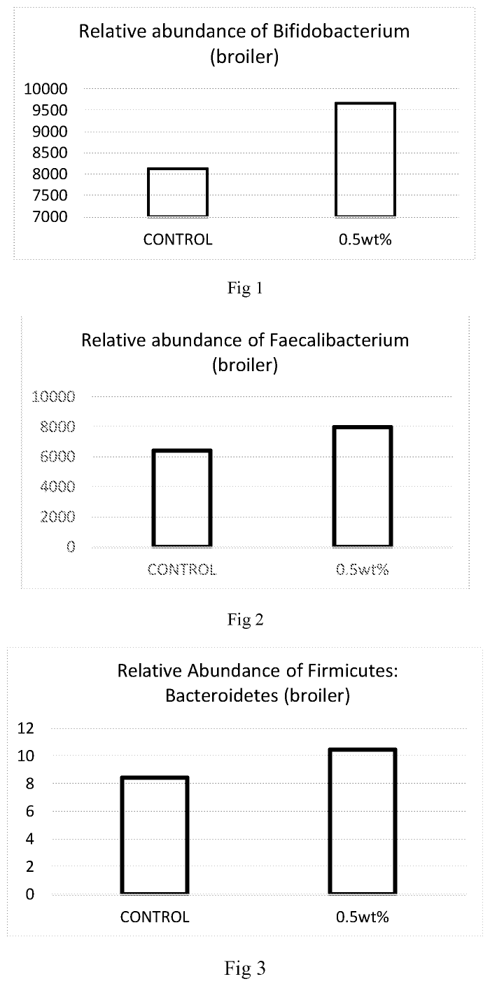

Referring to FIG. 2, Faecalibacterium, a butyrate producing bacterial genus associated with carbohydrate metabolism was also higher. Butyrate has been shown to be anti-inflammatory.

Referring to FIG. 3, the addition of the seaweed blend increased the ratio of Firmicutes to Bacteroides. Petersen et al., (2013) observed a positive correlation between weight gain and relative abundance of Firmicutes.

Swine (Pig) Trial 1

| Recipe C | wt % |
|---|---|
| Ulva lactuca | 65-75 |
| Sargassum | 15-25 |
| Gracilaria | 2-15 |
| Ascophyllum nodosum | 2-15 |
| Maerl | 0-7 |

A seaweed blend of brown, green and red seaweeds (above) was added to the feed (0.5 wt % of the feed) of commercial pigs post-weaning (pigs weaned at 21 days of age) for 6 weeks. Faecal samples were collected to assess changes in the bacterial community. Faecal samples were analysed by 16S RNA qPCR, cloning and sequencing (BaseClear NV).

As shown in FIG. 4, the relative abundance of lactobacillus species from the faeces of pigs fed diets containing the seaweed blend was higher (20% compared to 19%) compared to control pigs fed the same diet without the addition of the seaweed bend.

The inventors propose that an increase in Lactobaciillus provides a benefit. Lactobacillus species such as L. fermentum have been used as a growth-promoting feed supplement preventing and treating diarrhoea of weaned piglets and maximising average daily gain, crude protein apparent digestibility and serum specific IgG level.

As shown in FIG. 5, the relative abundance of Butyricicoccus species in the faeces of pigs fed diets containing the seaweed blend was higher than the control.

As shown in FIG. 6, the addition of the feed supplement increased the relative abundance of Firmicutes: Bacteroidetes. Petersen et al., (2013) observed a positive correlation between weight gain and relative abundance of Firmicutes.

Further Investigations

Studies of the GI microbiome (genetic footprint of the microbiota) have shown that domestic animals such as swine and poultry (chickens) possess a core microbiome, which changes in composition based on certain factors such as diet and other environmental factors. Liu et al., December 2015, BMC Complementary and Alternative Medicine 15(1):279 DOI: 10.1186/s12906-015-0802-5 investigated the prebiotic effects of cultivated red seaweed Chondrus crispus in diets fed to young rats using 16S rRNA sequencing to profile the colonic microbiome. This study successfully demonstrated that seaweed increased the relative abundance of Bifidobacterium breve and decreases the abundance of pathogenic species such as Clostridium septicum and Streptococcus pneumonia. However it does not demonstrate a consistent increase in a family of butyrate producing bacteria. This present invention uses 16S rRNA sequencing to gain a snapshot of the GI microbiome to consistently define changes at the bacterial family level.

The commensal bacteria in the GI of domestic swine and poultry are those that ferment complex carbohydrates in the colon and ceca, producing metabolic intermediaries such as short chain fatty acids (SCFA) as the end products of fermentation (Fouhse et al., Animal Frontiers, Volume 6, Issue 3, July 2016, Pages 30-36, https://doi.org/10.2527/af.2016-0031). One of these SCFAs, Butyric Acid or Butyrate is preferentially utilized by the GI mucosa cells, as a source of energy and as a metabolic signal resulting in several digestive and systemic effects on the hosts. The results explained below demonstrate consistent increases in bacterial families that have been shown to be the main butyrate producing families in the lower GI of pigs and poultry.

Investigating the Gastrointestinal Microbiome

The potential for using unique blends of red, brown and green seaweed species as natural prebiotic feed additives was investigated in swine and in poultry (broiler chickens). Unique blends of green, brown and red seaweeds were added to the daily ration at rates between 0.50 and 0.75% of the food.

Diets containing the seaweed blends were fed throughout the early growth stage to pigs and chickens. At the end of the test period fresh faecal samples (pigs) or ceca samples (chickens) were collected from several replicate pens into collection tubes containing a preservative buffer solution (RNA/DNA Shield® Zymo Research), which preserved the samples during storage and shipping. The microbiomes of the faecal and ceca samples were analysed by 16S RNA qPCR gene (V3-4), cloning and sequencing using the Illumina Miseq® platform (BaseClear NV).

These results clearly demonstrate that the use of seaweed blends of red, brown and green species, positively stimulated increases in commensal bacterial families that have been shown to be the dominant butyrate producing microorganisms in the gastrointestinal tract. The observed increases in these commensal bacteria may lead to increases in gastrointestinal health and digestive efficiency.

Summary of Findings: Swine (Pig) Trial 2

| Recipe C’ | wt % |
|---|---|
| Ulva lactuca | 65-75 |
| Sargassum and Ascophyllum nodosum | 2-25 |
| Gracilaria | 2-15 |
| Maerl | 0-7 |

Referring to FIG. 9, compared to the Control group, pigs consuming diets containing blend C′ had improved firmicutes to bacteroidetes ratio. Bacteria in the phyla Firmicutes are associated with a leaner and more feed-efficient pigs.

Referring to FIGS. 10 and 11, analysis of faecal bacterial community also revealed an increase in the abundance of the bacterial families Ruminoccocaceae and Lachnospiraceae, which are producers of butyrate, a short chain fatty acid with positive effects in the gastrointestinal tract.

Summary of Findings: Broiler (Chicken) Trial 2

| Recipe B’ | wt % |
|---|---|
| Ulva lactuca | 70-80 |
| Ascophyllum and Sargassum | 3-20 |
| Gracilaria | 3-8 |
| Maerl | 0-3 |

In a 42-day growth performance study, seaweed blend inclusion to a wheat-soyabean based diet at 0.5% of the feed significantly increased the Firmicutes to Bacteroidetes ratio in the ceca of broiler chickens. This ratio indicates whether the microbiome is healthy, displaying a higher abundance of Firmicutes or in dysbiosis (a higher or increased abundance of Bacteroidetes). The results are shown in FIG. 13.

Referring to FIGS. 14 and 15, the relative abundance of important, butyrate producing bacterial families including Ruminococcaceae and Lachnospiraceae were increased in the ceca.

1. (canceled)

2. A method comprising
providing a seaweed blend to an animal host, the animal host having a gastrointestinal (GI) microbiota; and
modifying the gastrointestinal (GI) microbiota of the animal host.

3. The method of claim 2, wherein the seaweed blend has a composition and is delivered to the animal host at a dosage; and the composition and/or the dosage of the seaweed blend is selected in order to target a specific modification of the GI microbiota.

4. The method of claim 3, comprising an initial step of analysing a sample from the host to determine the relative abundance and/or population of at least one microorganism.

5. The method of claim 2, wherein modifying the GI microbiota comprises modifying a community of bacteria.

6. The method of claim 5, wherein the community of bacteria comprises Firmicutes and Bacteriodetes and the seaweed blend modifies the ratio of Firmicutes to Bacteriodetes, such as increasing the ratio of Firmicutes to Bacteriodetes.

7. The method of claim 5, wherein the seaweed blend modifies the relative abundance of bacteria of one or more of Bacteroides, Clostridium, Faecalibacterium, Eubacterium, Ruminococcus, Peptococcus, Peptostreptococcus, Bifidobacterium, Prevotella, Butyricicoccus, Coprococcus, Prevotella, Escherichia and Lactobacillus, such as increasing the relative abundance of one or more of (i) Faecalibacterium; (ii) Bifidobacterium; (iii) Butyricicoccus; (iv) Coprococcus; and (v) Lactobacillus.

8. The method of claim 5, wherein the seaweed blend modifies the relative abundance of bacteria of one or more of Ruminococcaceae (such as Faecalibacterium, Ruminococcus, Lachnospiraceae, and Coprococcus and, Blautia); Peptococcus, Peptostreptococcus, Bifidobacteriaceae (such as Bifidobacterium); Lactobacillaceae (such as Lactobacillus); Clostridiaceae (such as Butyricicoccus); Prevotelleceae (such as Prevotella), Enterobacteriaceae (such as Escherichia).

9. The method of claim 5, wherein the seaweed blend increases the relative abundance of one or more of (i) Faecalibacterium; (ii) Bifidobacterium; (iii) Butyricicoccus; (iv) Coprococcus; and (v) Lactobacillus.

10. The use-OF method of claim 5, wherein the seaweed blend reduces the relative abundance of Prevotella and/or Escherichia

11. The method of claim 2, wherein the seaweed blend comprises a blend of green seaweed, brown seaweed and red seaweed.

12. The method claim 2, wherein the seaweed blend comprises 60 to 75 wt % green seaweed; 15 to 25 wt % brown seaweed; and 3 to 10 wt % red seaweed.

13. (canceled)

14. The method claim 2, wherein the seaweed blend comprises seaweed of at least three different genera.

15. The method claim 2, wherein the seaweed blend comprises (i) Ulva; (ii) Gracilaria; and (iii) Sargassum and/or Ascophyllum.

16. The method of claim 2, wherein the seaweed blend comprises (i) 65-80 wt % Ulva; (ii) 3-8 wt % Gracilaria; and (iii) 15-25 wt % Sargassum and/or Ascophyllum.

17. The method claim 2, wherein the seaweed blend comprises Recipe A′, Recipe B′, Recipe C′ and/or Recipe D′:

| | Recipe A’ | Recipe B’ | Recipe C’ | Recipe D’ |
|---|---|---|---|---|
| | (wt %) | (wt %) | (wt %) | (wt %) |
| Ulva lactuca | 70-80 | 70-80 | 65-75 | 55-70 |
| Ascophyllum and | 2-20 | 3-20 | 2-25 | 2-25 |
| Sargassum | | | | |
| Gracilaria | 3-8 | 3-8 | 2-15 | 3-10 |
| Maerl | 0-1 | 0-3 | 0-7 | 0-7 |

18. The method claim 2, wherein the host is a non-human animal.

19. The method of claim 2, wherein the host is selected from a bird, a pig, a sheep, a cow, a horse, and a companion animal.

20. The method of claim 2, wherein the host is a bird and the seaweed blend modifies
(i) a ratio of Firmicutes to Bacteroidetes;
(ii) relative abundance of Faecalibacterium; and/or
(iii) relative abundance of Bifidobacterium.

21. The method of claim 2, wherein the host is a pig and the seaweed blend modifies
(i) ratio of Firmicutes to Bacteroidetes;
(ii) relative abundance of Lactobaciillus;
(iii) relative abundance of Butyricicoccus;
(iv) relative abundance of Coprococcus and/or
(v) relative abundance of prevotella.

22. The method claim 2, wherein
(i) the seaweed blend is formulated as a tablet, a capsule or a pellet;
(ii) the seaweed blend is provided to the host in an amount of at least 0.5 wt % relative to a regular feed;
(iii) the seaweed blend is incorporated into a feed composition;
(iv) the seaweed blend is provided to the host daily for at least 4 weeks; and/or
(v) the host has a diet free of antibiotics and/or coccidiostat.
